(19) United States
(12) Patent Application Publication
(10) Pub. No.: US 2005/0221319 A1
(43) Pub. Date: Oct. 6, 2005

(54) Use of capturing probes for identifying nucleic acids

(75) Inventor: Kerstin Korn, Stockholm (SE)

Correspondence Address: ROTHWELL, FIGG, ERNST & MANBECK, P.C., 1425 K STREET, N.W., SUITE 800, WASHINGTON, DC, 20005, US

(73) Assignee: Gnothis Holding SA, Ecublens (CH)

(21) Appl. No.: 10/507,815

(22) PCT Filed: Mar. 14, 2003
(86) PCT No.: PCT/EP03/02713

§ 371 Date: May 4, 2005

(30) Foreign Application Priority Data
Mar. 14, 2002 (DE) 102113211

(51) Int. Cl. C12Q 1/68

(52) U.S. Cl. 435/6

(57) ABSTRACT

The invention relates to a method for determining a nucleic acid analyte in a sample with use of a hybridization probe able to bind to the analytes and of a capture probe able to bind to the hybridization probe. A reagent kit containing hybridization and capture probes is also provided. Method and reagent kit can be employed in particular for determining analytes which occur in only very low concentrations in the sample.

The invention relates to a method for determining a nucleic acid analyte in a sample with use of a hybridization probe able to bind to the analytes and of a capture probe able to bind to the hybridization probe. A reagent kit containing hybridization and capture probes is also provided. Method and reagent kit can be employed in particular for determining analytes which occur in only very low concentrations in the sample.

The use of hybridization probes for detecting nucleic acid target molecules as analytes in a sample has been known for a long time. The hybridization probe normally used is a probe which is complementary to a partial sequence of the analyte and is composed of nucleotide units or nucleotide analog units and which binds specifically to the analyte present in the sample and carries a detectable labeling group.

A particularly sensitive method for detecting analytes, including nucleic acid analytes, is so-called fluorescence correlation spectroscopy (FCS) which is described for example in EP-B-0 679 251. This method allows only a very small number of molecules in the sample to be detected and even allows detection of single molecules.

A problem—which exists generally in hybridization methods but especially in highly sensitive methods such as, for example, FCS—is a background signal which is produced by labeled hybridization probes not bound to the analyte. The intensity of this background signal is moreover increased by the presence of an excess of labeled hybridization probes compared with the analyte molecules present in the sample, which excess is necessary in many cases, for example for kinetic reasons or for the quantitative determination of analytes.

The signal derived from unbound hybridization probes can be reduced by employing so-called molecular beacons which normally comprise a hairpin structure consisting of a stem with two mutually complementary sequence sections and of a loop, and a fluorescence labeling group and a quencher group. The stem sequence which is unable to bind to the target molecule serves to hold the fluorescence labeling group in proximity to the quencher group when the probe is not bound to the analyte. In this way, photons absorbed by the fluorescence labeling group are not emitted as fluorescence photons, rather the energy is transferred to the quencher group. The loop sequence is complementary to the target molecule to be detected. When the probe binds to the target molecule, the loop is opened and hybridizes with the target molecule, so that the fluorescence labeling group is removed from proximity to the quencher group. It is thus possible for fluorescence to occur and be measured.

One object on which the present invention is based was thus to avoid at least in part the abovementioned disadvantages in a method for determining nucleic acid analytes.

This object is achieved by selectively attenuating or reducing a background signal which is derived from labeling groups in the sample which are not bound to the analyte, through use of a capture probe which is able to bind to the hybridization probe but is not covalently associated therewith. This leads to an improvement in the ratio between the measured signal derived from a labeling group bound to the analyte, and the background signal, which is preferably a factor of at least 2 and particularly preferably a factor of at least 5. A combination comprising a hybridization probe and a capture probe able to bind to the hybridization probe is employed for this purpose, where the capture probe carries a capture group and, through the binding to a free, i.e. not analyte-bound, hybridization probe brings about a reduction in the signal of the labeling group thereof. In order not to impair the binding of the hybridization probe to the analyte, hybrids between capture probes and hybridization probes must have a lower melting point than hybrids between analytes and hybridization probes.

One aspect of the invention is thus a method for determining a nucleic acid analyte in a sample, comprising the steps:
(a) provision of a sample which may contain the analyte,
(b) provision of at least one hybridization probe which is able to bind to the analyte and carries a labeling group,
(c) provision of at least one capture probe able to bind to the hybridization probe, where the capture probe carries at least one capture group which, on binding to the hybridization probe, brings about a reduction in the signal derived from the labeling group, and where a hybrid between capture probe and hybridization probe has a lower melting point than a hybrid between analyte and hybridization probe,
(d) contacting sample and hybridization probe under first hybridization conditions with which a stable hybrid is possible between hybridization probe and analyte, but with which no stable hybrid is possible between hybridization probe and capture probe,
(e) contacting sample, hybridization probe and capture probe under second hybridization conditions with which a stable hybrid is possible between hybridization probe not bound to the analyte, and capture probe, and where through binding of the hybridization probe to the capture probe a reduction is brought about in the signal derived from the labeling group thereof, and
(f) detection of the binding of the hybridization probe to the analyte.

The nucleic acid analytes determined by the method, e.g. DNA or/and RNA, may be derived from biological samples, in particular from mammals such as humans, but also other organisms, e.g. microorganisms. It is also possible in addition to detect analytes which have been produced in vitro from biological samples, e.g. cDNA molecules which have been prepared by reverse transcription from mRNA. The sample used for the method is preferably derived from an organism, e.g. from tissue or body fluid of an organism, in particular from a human.

The method can be employed for example to analyze gene expression, e.g. to establish a gene expression profile or to analyze mutations, e.g. single nucleotide polymorphisms (SNP). The method is also suitable, however, for determining enzymatic reactions on nucleic acids, for example for determining nucleic acid amplification reactions, especially in one or more thermocycling processes.

The hybridization probes employed for the method of the invention are preferably oligonucleotides, in particular DNA molecules. However, it is also possible to employ appropriate nucleic acid analogs such as, for example, peptide-nucleic acids (PNA), locked nucleic acids (LNA) or other nucleotide analogs. The length of the hybridization probes can in principle be chosen as for known probes and is normally in the range from 15 to 200 nucleotides.

The hybridization probes carry a labeling group which can produce a signal detectable by suitable methods. The labeling group preferably produces a signal detectable by optical methods. The labeling group is particularly preferably a fluorescence labeling such as, for example, fluorescein, rhodamine, phycoerythrin, CY3, CY5 or derivatives thereof.

An essential feature of the method of the invention is the use of capture probes carrying a capture group. The capture probes are able under suitable hybridization conditions to bind to the hybridization probes but are not covalently associated therewith. The capture probes are—just like the hybridization probes—preferably oligonucleotides, in particular DNA molecules. However, it is also possible to employ appropriate nucleic acid analogs as mentioned above. The length of the capture probes can be chosen to correspond to the length of the hybridization probes.

In a first preferred embodiment, it is possible to use a quencher group which at least partly quenches the signal of the labeling group, for example of a fluorescence labeling group, as capture group. If a quencher group is present in spatial proximity to a fluorescence labeling group, photons absorbed by the fluorescence labeling group are not emitted as fluorescence photons, rather the energy is transferred to the quencher group. In this embodiment, therefore, it is expedient to choose the positions of the labeling group on the hybridization probe and of the quencher group on the capture probe so that the two groups are in spatial proximity in a hybrid of hybridization probe and quencher group. Thus, for example, possible dispositions are the labeling group at the 5′ end of the hybridization probe and the quencher group at the 3′ end of the capture probe or the labeling group at the 3′ end of the hybridization probe and the quencher group at the 5′ end of the capture probe. Examples of quencher groups able to quench at least partly the signal produced by a fluorescence labeling group are disclosed in U.S. Pat. No. 5,607,834.

In a further preferred embodiment it is possible to use as capture groups also solid phase binding groups which bring about a selective removal of the labeling group not bound to the analyte, by immobilization on a suitable solid phase. Examples of solid phase binding groups and solid phases which can be bound thereto are binding partners able to undergo high-affinity interactions with one another, such as, for example, biotin (and biotin analogs) and streptavidin or avidin, or an antigen or a hapten and an antibody, or a sugar and a lectin. In a particularly preferred embodiment a biotinylated capture probe and a solid phase coated with streptavidin or avidin are used. The solid phase can be of any type, for example the wall of a reaction vessel, a particulate solid phase such as, for example, microbeads, or a membrane through which the sample flows.

An essential feature of the method of the invention is that a hybrid between capture probe and hybridization probe has a lower melting point than a hybrid between analyte and hybridization probe. This melting point difference is, under assay conditions, beneficially at least 1° C., preferably at least 2° C. and particularly preferably at least 5° C. The different stability of the hybrids can be achieved for example through the capture probe containing a shorter region complementary to the hybridization probe than the analyte or/and containing at least one mismatch (i.e. a base which is not complementary to the hybridization probe) in the region complementary to the analyte.

In step (d) of the method of the invention there is contacting of sample and hybridization probe (in the presence or absence of the capture probe) under first hybridization conditions with which a stable hybrid is possible between hybridization probe and analyte, but with which no stable hybrid is possible between hybridization probe and capture probe. This first hybridization step removes by reaction those hybridization probe molecules able to hybridize with the analyte molecules present in the sample, while the surplus hybridization probe molecules—in the presence or in the absence of the capture probe—remain in unbound form. During the second hybridization step (e) in the presence of sample, hybridization probe and capture probe, second hybridization conditions are set, e.g. by reducing the temperature, with which a stable hybrid is also possible between hybridization probe molecules not bound to the analyte, and capture probe molecules.

It is expedient to use the capture probes in a sufficient amount for hybridization probes which are not bound to the analyte to be substantially quantitatively bound, e.g. in a molar excess of preferably at least ≧1.5:1, particularly preferably ≧2:1, in relation to the hybridization probe molecules.

The capture of the hybridization probes selectively reduces the background signal of the labeling groups not bound to the analyte for example by quenching or/and by removal for solid-phase binding. In the detection of the binding of the hybridization probe to the analyte in (f) of the method of the invention, it is thus possible to achieve a significant reduction in the nonspecific background. A further advantage is the avoidance of nonspecific binding of hybridization probes which are not bound to the analyte to further nucleic acid molecules present in the sample.

In a particularly preferred embodiment of the method at least two hybridization probes which are able to bind to the analyte and each of which carry different labeling groups, and in each case one capture probe per hybridization probe, are used. In this case it is possible to detect the analyte by simultaneous binding of the at least two hybridization probes.

Different hybridization probes comprise labeling groups, preferably fluorescence labeling groups, which differ in at least one measured parameter, in particular selected from emission wavelength and duration of fluorescence.

The determination may include detection of an energy transfer between the labeling groups of the first and second probe. For example, one probe comprises a donor labeling group and the other probe comprises an acceptor labeling group, in which case an energy transfer can take place between donor and acceptor labeling groups. Suitable combinations of donor and acceptor labeling groups for such an energy transfer (FRET) are known to the skilled worker (see, for example, U.S. Pat. No. 4,996,143).

The method of the invention may, however, also include a so-called cross-correlation determination in which at least two different labelings, especially fluorescence labelings, are employed, and the correlated signal thereof is determined. The fundamental procedure for such a cross-correlation determination is described for example in Schwille et al. (Biophys. J. 72 (1997), 1878-1886) and Rigler et al. (J. Biotechnol. 63 (1998), 97-109).

One possibility for reducing interference in cross-correlation is to carry out a time-resolved determination by time gating. In this case, the at least two spectrally different fluorescence labeling groups bound to the analyte are excited repetitively at a high impulse rate which is of the order of magnitude of the delay of the detector, e.g. in the range from 10 to 0.1 MHz (equivalent to clock intervals from 0.1 to 10 μs). It is moreover possible by appropriate electronic background analysis with calculation of a correlation or/and coincidence curve to discriminate “correctly” occurring correlation signals from those signals produced by unwanted interference (crosstalk) of the two labeling groups.

The method of the invention is preferably carried out with low concentrations of the analyte present in the sample or of the hybridization probes and with a very low sample volume. The sample volume is preferably in the region of ≦10⁻⁶ l and particularly preferably ≦10⁻⁸ l. Such samples can be determined by using as support a microwell structure with a plurality of wells to receive sample liquid, the individual wells having, for example, a diameter between 10 and 1000 μm. Suitable microstructures are described, for example, in DE 100 23 421.6 and DE 100 65 632.3. The support may additionally comprise temperature-control elements, e.g. Peltier elements, which make it possible to control the temperature of the support or/and of individual sample containers therein. The support used for the method is expediently equipped in such a way that it makes optical detection of the sample possible. A support which is optically transparent at least in the region of the sample containers is therefore preferably used. The support may moreover either be completely optically transparent or contain an optically transparent base and an optically opaque covering layer with recesses in the sample containers. Suitable materials for supports are, for example, composite supports made of metal (e.g. silicon for the covering layer) and glass (for the base). Supports of this type can be produced for example by applying a metal layer with predetermined recesses for the sample containers to the glass. An alternative possibility is to employ plastic supports, e.g. made of polystyrene or acrylate- or methacrylate-based polymers. It is additionally preferred for the support to have a covering for the sample containers in order to provide a closed system which is substantially isolated from the surroundings during the measurement.

A further possibility is to generate electric fields in the support, especially in the region of the sample containers, in order to achieve a concentration of the analytes to be determined in the measured volume. Examples of electrodes which are suitable for generating such electric fields are described for example in DE 101 03 304.4.

The analyte is particularly preferably detected by fluorescence correlation spectroscopy (FSC). In this case, the measurement of one or a few analyte molecules preferably takes place in a measured volume which is part of the sample volume, where the concentration of the analyte molecules to be determined is preferably ≦10⁻⁶ mol/l, and the measured volume is preferably ≦10⁻¹⁴ l. The measured signals derived from the labeling groups of the hybridization probes are ascertained in this case by luminescence measurement. Reference is made to EP-B-0 679 251 for specifics concerning the details of the apparatus for the method and devices suitable for carrying out the method.

An alternative possibility is for the detection also to take place by time-resolved decay measurement, a so-called time/gating, where the signal of the labeling groups, e.g. the fluorescence, are excited within the measured volume, and subsequently the opening of the detection interval at the detector preferably takes place in a time interval of ≧100 ps. This method of measurement is described for example by Rigler et al. in “Ultrafast Phenomena” D. H. Auston, editor, Springer 1984.

In a particularly preferred embodiment of the method, a confocal single-molecule analysis is carried out in such a way that the distance between the measured volume in the sample liquid and the system for focusing the light source used to excite fluorescence labeling groups in the sample liquid is ≧1 mm and the sample liquid a light source is thermally insulated in particular from the focusing system. A method of this type and a device suitable for this purposes is described for example in DE 101 11 420.6. It is possible by thermal insulation of the sample liquid from components of the apparatus for the temperature of the sample to be adjusted independently and varied during the method. It is thus possible to carry out temperature-variable processes, e.g. determination of nucleic acid hybridization melting curves or determination of nucleic acid amplification reactions.

The support is preferably a microstructure having a plurality of, preferably at least 10², containers to receive a sample liquid, it being possible for the sample liquid in the separate containers to be derived from one or more sources. The introduction of the sample liquid into the containers of the support can take place for example by means of a piezoelectric liquid-dispensing device.

The containers of the support are equipped in such a way that binding of the detection reagent with the analyte in solution is made possible. The containers are preferably wells in the support surface, it being possible for these wells in principle to have any shape, e.g. circular, square, rhomboid etc. The support may also comprise 10³ or more separate containers.

The optical excitation unit comprises a strongly focused light source, preferably a laser beam, which is focused by means of appropriate optical units onto the measured volume in the sample liquid. The light source may also contain two or more laser beams, each of which is then focused onto the measured volume by different systems before entering into the sample liquid. The detection unit may contain for example a fiber-coupled avalanche photodiode detector or an electronic detector. However, it is also possible to use excitation or/and detection matrices consisting of a dot matrix of laser dots, generated by a diffraction system or a quantum well laser, and of a detector matrix, generated by an avalanche photodiode matrix or electronic detector matrix, e.g. a CCD camera.

In a preferred embodiment, the light source includes one or more laser beams which are split into multiple foci by passing through one or more diffraction elements, as described in DE 101 26 083.0.

The support can be provided in prefabricated form, in which case luminescence-labeled detection reagents, preferably luminescence-labeled hybridization probes or primers, are introduced into a plurality of separate containers of the support. The support containing the detection reagents is then beneficially dried.

In a preferred embodiment of the invention there is provision of a prefabricated support which contains a large number of separate, e.g. 100, containers into each of which different detection reagents, e.g. reagents for detecting a nucleic acid hybridization, such as primers or/and probes, are introduced. This support can then be charged with a sample derived from an organism to be investigated, e.g. from a human patient, so that different analytes from a single sample can be determined in the respective containers. Supports of this type can be used for example for constructing a gene expression profile, e.g. for diagnosing diseases, or for determining nucleic acid polymorphisms, e.g. for detecting a particular genetic predisposition.

Finally, the invention relates to a reagent kit for detecting nucleic acid analytes comprising
(a) at least one hybridization probe which is able to bind to an analyte and carries a labeling group, and
(b) at least one capture probe able to bind to the hybridization probe, where the capture probe carries at least one capture group which, on binding to the hybridization probe, brings about a reduction in the signal derived from the labeling group, and where a hybrid between capture probe and hybridization probe has a lower melting point than a hybrid between analyte and hybridization probe.

This reagent kit is preferably employed in a detection method as described above.

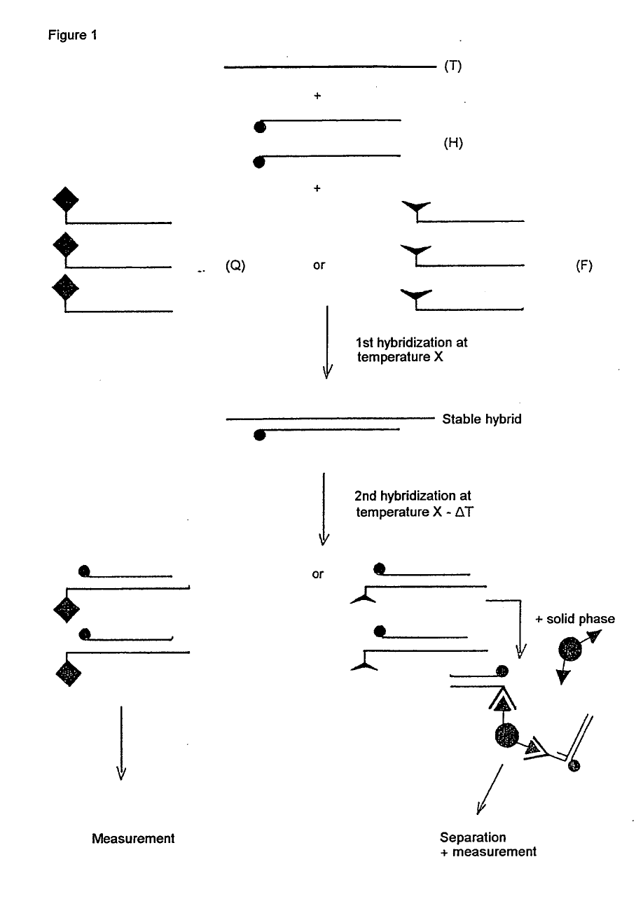

FIG. 1 shows a preferred embodiment of the present invention. A sample with a target nucleic acid (T) contained therein is contacted with a hybridization probe (H) and a capture probe comprising a quencher group (O) or a solid phase binding group (F). Firstly there is a first hybridization at a temperature X at which a stable hybrid is produced between target nucleic acid (T) and hybridization probe (H), but at which no stable hybridization products are possible between the capture probes (Q or F) and the hybridization probe (H).

After a sufficient incubation time, a second hybridization takes place at a temperature X−^{Δ}T, in which case stable hybridization products are possible between hybridization probe (H) and the capture probes (Q and F). When capture probes (F) having solid phase binding groups are used, it is necessary to separate the second hybridization products from the first hybridization products by solid phase binding, e.g. by adding suitable coated beads and subsequent centrifugation.

Finally, detection of the first hybridization product of target nucleic acid (T) and hybridization probe (A) is possible without interference with this detection by excess free hybridization probe molecules.

1. A method for determining a nucleic acid analyte in a sample, comprising the steps:
(a) provision of a sample which may contain the analyte,
(b)-provision of at least one hybridization probe which is able to bind to the analyte and carries a labeling group,
(c) provision of at least one capture probe able to bind to the hybridization probe, where the capture probe carries at least one capture group which, on binding to the hybridization probe, brings about a reduction in the signal derived from the labeling group, and where a hybrid between capture probe and hybridization probe has a lower melting point than a hybrid between analyte and hybridization probe,
(d) contacting sample and hybridization probe under first hybridization conditions with which a stable hybrid is possible between hybridization probe and analyte, but with which no stable hybrid is possible between hybridization probe and capture probe,
(e) contacting sample, hybridization probe and capture probe under second hybridization conditions with which a stable hybrid is possible between hybridization probe not bound to the analyte, and capture probe, and where through binding of the hybridization probe to the capture probe a reduction is brought about in the signal derived from the labeling group thereof, and
(f) detection of the binding of the hybridization probe to the analyte.

2. The method as claimed in claim 1, characterized in that hybridization probes which carry a fluorescence labeling group are used.

3. The method as claimed in claim 1, characterized in that a quencher group which at least partly quenches the signal of the labeling group is used as capture group.

4. The method as claimed in claim 1, characterized in that a solid phase binding group is used as capture group, and the hybridization probe bound to the capture probe is removed by immobilization on a solid phase.

5. The method as claimed in claim 4, characterized in that a biotinylated capture probe and a streptavidin- or avidin-coated solid phase are used.

6. The method as claimed in claim 1, characterized in that the capture probe has a shorter region complementary to the hybridization probe than the analyte or/and has at least one mismatch in the region complementary to the analyte.

7. The method as claimed in claim 1, characterized in that the melting point difference of the hybrid between capture probe and hybridization probe and of the hybrid between analyte and hybridization probe is at least 1° C., preferably at least 2° C. and particularly preferably at least 5° C. under assay conditions.

8. The method as claimed in claim 1, characterized in that at least two hybridization probes which are able to bind to the analyte and each of which carry different labeling groups, and in each case one capture probe per hybridization probe, are used.

9. The method as claimed in claim 8, characterized in that the analyte is detected by simultaneous binding of the at least two hybridization probes.

10. The method as claimed in claim 1, characterized in that step (f) includes a detection by confocal detection.

11. The method as claimed in claim 10, characterized in that the detection includes a single molecule detection.

12. The method as claimed in claim 10, characterized in that step (f) includes a detection by fluorescence correlation spectroscopy or/and time gating.

13. The method as claimed in claim 10, characterized in that a single light source is used to excite the labeling group(s).

14. The method as claimed in claim 10, characterized in that a plurality of light sources is used to excite the labeling group(s).

15. The method as claimed in claim 2 characterized in that the light source(s) used to excite the labeling groups is (are) beamed into the sample in the form of repetitive impulses.

16. The method as claimed in claim 15, characterized in that the impulse rate is in the range from 10 to 0.1 MHz.

17. A reagent kit for detecting nucleic acid analytes, comprising:
(a) at least one hybridization probe which is able to bind to an analyte and carries a labeling group, and
(b) at least one capture probe able to bind to the hybridization probe, where the capture probe carries at least one capture group which, on binding to the hybridization probe, brings about a reduction in the signal derived from the labeling group, and where a hybrid between capture probe and hybridization probe has a lower melting point than a hybrid between analyte and hybridization probe.
